(19) United States
(12) Patent Application Publication
(10) Pub. No.: US 2015/0079062 A1
(43) Pub. Date: Mar. 19, 2015

(54) METHOD FOR HARVESTING, PROCESSING, AND STORAGE OF PROTEINS FROM THE MAMMALIAN FETO-PLACENTAL UNIT AND USE OF SUCH PROTEINS IN COMPOSITIONS AND MEDICAL TREATMENT

(71) Applicants: Patrick J. Casey, Kumeu (NZ); Murray Hoare, Kumeu (NZ)

(72) Inventors: Patrick J. Casey, Kumeu (NZ); Murray Hoare, Kumeu (NZ)

(21) Appl. No.: 14/551,612

(22) Filed: Nov. 24, 2014

Related U.S. Application Data
(63) Continuation-in-part of application No. 14/330,818, filed on Jul. 14, 2014.
(60) Provisional application No. 61/845,682, filed on Jul. 12, 2013.

(51) Int. Cl. A61K 35/54; A61K 38/17; A01N 1/02; A61K 35/50

(52) U.S. Cl. CPC A61K 35/54; A61K 35/50; A61K 38/1709; A01N 1/0284
USPC 424/94.2; 424/582; 424/583; 424/551; 424/553; 424/559; 424/570; 424/571

(57) ABSTRACT

The invention provides a method of harvesting, processing and storing a plurality of proteins from a mammalian feto-placental unit. The method includes dissecting a mammalian uterus to harvest at least one component of the mammalian feto-placental unit; blast freezing the component; and storing the blast frozen component; wherein the blast frozen component includes the proteins. In one embodiment, the method includes lyophilizing the blast frozen component to remove at least some water from the blast frozen component thereby creating a freeze-dried form; and storing the lyophilized component; wherein the lyophilized component includes the proteins. The invention also provides a composition including proteins from at least one lyophilized, blast frozen component of a harvested mammalian feto-placental unit reconstituted with a fluid. The invention further provides method of treatment of a disease or aging in a mammalian subject including administering to the mammalian subject a plurality of proteins from at least one blast frozen component harvested from a mammalian feto-placental unit; and reducing an accumulation of at least one intracellular protein in the subject.

### STATEMENT REGARDING FEDERALLY SPONSORED RESEARCH OR DEVELOPMENT

Not Applicable

### BACKGROUND OF THE INVENTION

The present invention broadly relates to methods of harvesting mammalian feto-placental proteins and in particular, chaperone proteins, for use in compositions and medical therapies for the treatment of disease or aging in mammals, and in particular, to humans.

Aging, which is both chronological and determined by cellular processes, is associated with an increase in disease and pathological processes. The causes of aging are unknown, but recent reviews correlate aging in humans with the instability of mitochondria leading to an increase in intra-cellular reactive oxygen species (ROS), potential shortening of the telomere, protein dysfunction and/or cellular death. The increase in ROS in turn appears to adversely affect DNA, mRNA, and protein synthesis, folding, transport and degradation. The culmination of these events in turn leads to apoptosis, i.e., cellular “suicide”, followed by necrosis, i.e., cellular death.

Some research has focused on mechanisms which initiate or allow the up-regulation of intra-cellular ROS to determine whether the mechanisms are sudden or cumulative and whether the mechanism can be slowed to any extent. Research has also investigated the role of chaperone proteins in responding to increased ROS. Notably, aging appears to decrease the intra-cellular and extra-cellular levels of chaperone proteins. For the purposes of this application, chaperone proteins, which include pharmacological chaperones, pharmacoperones, and pharma-cochaperones, are defined as target-specific, small molecules that bind to their target proteins to facilitate biogenesis and/or prevent and/or correct protein misfolding. Pharmacological chaperoning: a primer on mechanism and pharmacology, Leidenheimer Nancy J., Ryder Katelyn G., Pharmacol Res. 2014 May;83:10-9. doi: 10.1016/j.phrs.2014.01.005. Epub 2014 Feb. 14. Chaperone protein targets include enzymes, receptors, transporters, and ion channels. Id. Chaperone proteins prevent the accumulation of misfolded proteins by promoting their refolding, degeneration and/or exocytosis. Keep your heart in shape: molecular chaperone networks for treating heart disease, Tarone Guido, Brancaccio Mara, Cardiovasc Res. 2014 Jun. 1;102(3):346-61. doi: 10.1093/cvr/cvu049. Epub 2014 Feb 28., PMID:24585203 [PubMed—in process]. Chaperone proteins also play a role in intracellular signaling by controlling conformational changes required for the activation and deactivation of signaling proteins and assembly in signalosome complexes. Id.

Today, chaperone proteins are not readily available for medical research and possible medical therapies, however. Thus, there is a need for methods of harvesting, processing and storing chaperone proteins. In addition, there is a need to develop compositions and medical therapies which make use of chaperone proteins in the treatment of disease or aging, and in particular, disease related to protein dysfunction.

### BRIEF SUMMARY OF THE INVENTION

In one aspect, the invention provides a method of harvesting, processing and storing a plurality of proteins from a mammalian feto-placental unit. The method includes dissecting a mammalian uterus to harvest at least one component of the mammalian feto-placental unit; blast freezing the component; and storing the blast frozen component; wherein the blast frozen component includes the plurality of mammalian feto-placental unit proteins. In one embodiment, the method includes lyophilizing the blast frozen component to remove at least some water from the blast frozen component thereby creating a freeze-dried form; and storing the lyophilized component; wherein the lyophilized component includes the mammalian feto-placental unit proteins. In an additional embodiment, the mammalian feto-placental unit proteins include at least one essential fragment of at least one of the mammalian feto-placental unit proteins. In a further additional embodiment, the mammalian feto-placental unit proteins include a plurality of chaperone proteins.

In another embodiment, the mammalian feto-placental unit proteins are selected from the group consisting of Serum albumin, Actin, cytoplasmic 1, I alpha globin, Hemoglobin fetal subunit beta, Vimentin, Beta globin chain, TPM1, Annexin A2, Protein disulfide isomerase family A member 3, Alpha-2-HS-glycoprotein, Fatty acid binding protein 5, Cofilin-1, 78 kDa glucose-regulated protein, Gelsolin isoform b, Beta-A globin chain, Glyceraldehyde-3-phosphate dehydrogenase, Heat shock protein alpha, Heat shock protein 70, Peptidylprolyl isomerase A, 14-3-3 protein zeta/delta, Histone H3, Peroxiredoxin 2, Cathepsin D, Uterine milk protein, Tubulin beta chain, Myosin light chain 6, Endoplasmic reticulum protein 29, Tubulin alpha chain, Solute carrier family 2, facilitated glucose transporter member 1, Alpha-1-antitrypsin transcript variant 1, Heat shock protein 10, Pregnancy-associated glycoprotein 3, Hemoglobin subunit beta, Isocitrate dehydrogenase [NADP] cytoplasmic, Elongation factor-1 alpha, Phosphoglycerate kinase, 14-3-3 protein epsilon, Putative tropomyosin, Tumor protein translationally-controlled 1, Galectin-1, Transaldolase 1, Pregnancy-associated glycoprotein 4, Pregnancy-associated glycoprotein 1, Sodium/potassium-transporting ATPase subunit alpha-1, Lamin B1, pregnancy-associated glycoprotein 6, 14-3-3 protein beta/alpha, metallopeptidase inhibitor 2, fatty acid binding protein 5, Myosin regulatory light chain MRCL3, Transferrin, Enolase 1, Cathelicidin-1, 6-phosphogluconate dehydrogenase decarboxylating, Elongation factor-1 alpha, ATP-citrate synthase, Ribosomal protein S8, Pyruvate kinase, Pre-mRNA splicing factor SRP20-like protein, alpha 2, 5 prime, Malate dehydrogenase, Cystatin-B, Chorionic somatomammotropin hormone, Carbonic anhydrase 2, SLC25A6, Decorin, 60S ribosomal protein L6, Protein disulfide isomerase-associated 4, Pregnancy-associated glycoprotein 11, Prostaglandin F synthase, Integrin beta-1, H+ transporting ATP synthase subunit D, RHOA, Adenylate kinase, Lactate dehydrogenase A, RAB10, Glucose-6-phosphate 1-dehydrogenase, Elongation factor 1-delta, Ribosomal protein S17, Insulin-like growth factor-binding protein-7, Ribosomal protein L19, ATP synthase alpha subunit, RAC1, Calpain II 80 kDa subunit, Secreted phosphoprotein 24, CD9 antigen, Aspartate aminotransferase, DNA mismatch repair protein MutL, Ribosomal protein S6, 14-3-3 protein gamma LDHA protein, Putative peptidase, Myosin light chain kinase, smooth muscle, cAMP-dependent protein kinase regulatory subunit alpha 1, Elongation factor 1-alpha, Actin, GNAZ, Eukaryotic translation initiation factor 5A, Mitochondrial bifunctional methylenetetrahydrofolate dehydrogenase/cyclohydrolase, Solute carrier family 2, facilitated glucose transporter member 3, Thioredoxin, ADP-ribosylation factor 1, NADH dehydrogenase (Ubiquinone) 1 beta, Proteasome subunit alpha type, 2, Gamma fibrinogen, Putative H-ATPase subunit B, Proteasome subunit alpha type, 60S ribosomal protein L10, 14-3-3 protein sigma, Chaperone protein DnaK, Ribosomal protein s15, Putative uncharacterized protein, Aspartyl-tRNA synthetase, and Proteasome subunit alpha.

In another embodiment, the method includes using at least a portion of the mammalian feto-placental unit proteins for medicinal purposes. In an additional embodiment, the step of using at least a portion of the mammalian feto-placental unit proteins for medicinal purposes includes treating for cellular repair in a mammalian subject. In another embodiment, the step of using at least a portion of the mammalian feto-placental unit proteins for medicinal purposes includes treating for a disease or aging in a mammalian subject. In still other embodiments, the treating step includes administering the portion of the mammalian feto-placental unit proteins to the mammalian subject with at least one of a sublingual procedure, an intra-ocular procedure, an intra-rectal procedure, and an intra-gastro-intestinal procedure.

In an additional embodiment, the treating step includes reconstituting the portion of the mammalian feto-placental unit proteins of the lyophilized component with a fluid; and administering the reconstituted portion of the mammalian feto-placental unit proteins to the mammalian subject. In another embodiment, the fluid is an oil. In further additional embodiments, the administering step includes administering the reconstituted portion of the mammalian feto-placental unit proteins to the mammalian subject through a procedure selected from the group consisting of an oral administration, a rectal administration, a cutaneous administration, a subcutaneous administration, an intravenous injection, and an intramuscular injection. In a preferred embodiment, the administering procedure is a cutaneous administration

In another embodiment, the component is selected from the group consisting of a placenta-cord fetal component, a liver component, a spleen component, a whole brain component, an ocular component, a gastro-intestinal component, a female specific component, and a male specific component.

In another aspect, the invention provides a composition. The composition includes a plurality of mammalian feto-placental unit proteins from at least one lyophilized, blast frozen component of a harvested mammalian feto-placental unit. In one embodiment, the mammalian feto-placental unit proteins include at least one essential fragment of at least one of the mammalian feto-placental unit proteins. In another embodiment, the mammalian feto-placental unit proteins include a plurality of chaperone proteins. In yet another embodiment, the lyophilized, blast frozen component is selected from the group consisting of a placenta-cord fetal component, a liver component, a spleen component, a whole brain component, an ocular component, a gastro-intestinal component, a female specific component, and a male specific component.

In an additional aspect, the invention provides a method of treatment of a disease or aging in a mammalian subject including administering to the mammalian subject a plurality of mammalian feto-placental unit proteins from at least one blast frozen component harvested from a mammalian feto-placental unit; and reducing an accumulation of at least one intracellular protein in the mammalian subject. In one embodiment, the mammalian feto-placental unit proteins include at least one essential fragment of at least one of the mammalian feto-placental unit proteins. In another embodiment, the mammalian feto-placental unit proteins include a plurality of chaperone proteins. In yet another embodiment, the step of reducing the accumulation of the intracellular protein comprises at least one of folding at least a portion of the intracellular protein, refolding at least a portion of the intracellular protein, degrading at least a portion of the intracellular protein and transferring at least a portion of the intracellular protein across a cellular membrane.

### BRIEF DESCRIPTION OF THE SEVERAL VIEWS OF THE DRAWINGS

Further features of the present invention will become apparent from the following description of embodiments thereof, given by way of example only, with reference to the accompanying drawings, in which:

FIG. 1 shows the steps of the method according to an embodiment of the invention;

FIG. 2 shows the steps of the method according to an embodiment of the invention;

FIG. 3 shows the steps of the method according to an embodiment of the invention;

FIG. 4 shows the steps of the method according to an embodiment of the invention;

FIG. 5 shows schematically the mis-folding and refolding of a protein, as described in the prior art;

FIG. 6 shows schematically the degradation of a protein by a chaperone protein, as described in the prior art;

FIG. 7 shows schematically cell apoptosis, as described in the prior art; and

FIG. 8 shows the steps of the method according to an embodiment of the invention;

### DETAILED DESCRIPTION OF THE INVENTION

This application is a continuation-in-part of U.S. application Ser. No. 14/330,818 filed on Jul. 14, 2014 which claims priority to the previously filed U.S. Provisional Application No. 61/845,682 filed on Jul. 12, 2013 the entire contents of each of which is hereby incorporated by reference for all purposes.

The present invention relates to methods for harvesting proteins or essential fragments thereof, and in particular chaperone proteins, from the mammalian feto-placental unit, and the use of such mammalian feto-placental unit proteins in compositions and medical therapies for the treatment of disease and aging. For the purposes of the present application, the “essential fragment” of a protein of a mammalian feto-placental unit is defined as the portion of the protein which is capable of at least one of the reversal of cell apoptosis, the repair of cells, the regeneration of cells, and the regulation of protein folding, re-folding, transportation and degradation, through for example, the Ubiquitin Protease Pathway (UPP).

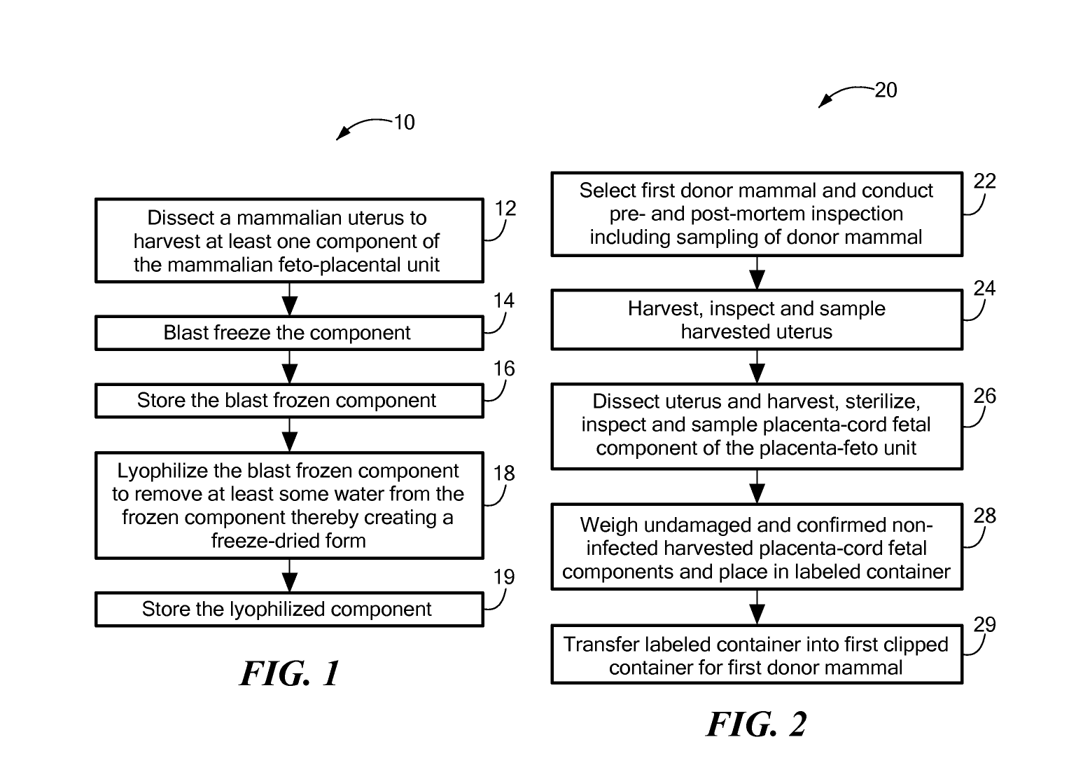

In one aspect, the invention provides a method 10 of harvesting and processing proteins from a mammalian feto-placental unit, as shown in FIG. 1. The method includes the steps of dissecting a mammalian uterus to harvest at least one component of the mammalian feto-placental unit, as shown in step 12; and blast freezing the component, as shown in step 14; wherein the component includes the proteins. In one embodiment, the method includes storing the blast frozen component, as shown in step 16. In another embodiment, the method includes lyophilizing the blast frozen component to remove at least some water from the frozen component thereby creating a freeze-dried form, as shown in step 18; and storing the lyophilized component, as shown in step 19.

In further embodiments, the invention provides the method 20 shown in FIG. 2. A female pregnant mammal, such as, for example, a pregnant ewe, is selected as a first donor mammal for supplying a uterus, as shown in step 22. A registered veterinarian conducts a pre-mortem and post-mortem examination of the selected first donor mammal to ensure that the donor is healthy and free of any visible disease. For example, the veterinarian inspects for muscular wasting which in an ovine could indicate parasitism or Johnes disease. The registered veterinarian also inspects for normal motor-function to rule out any central nervous system disease, such as, for example, aberrant larval migration, spinal cord and/or brain infection including infection in the middle and inner ear. The registered veterinarian also conducts a post-mortem examination of the selected first donor mammal to confirm the health and absence of disease in the donor mammal. The veterinarian can inspect for, for example, enlarged kidneys and nephrosis which could indicate a clostridial type infection. Core samples are taken from the liver and/or spleen of the first donor mammal for bacterial and viral isolation. For purposes of the present invention, only tissue negative to bacterial and viral isolation are harvested.

The uterus is harvested and inspected by the registered veterinarian to confirm the absence of lesions and/or other defects in the uterus, as shown in step 24. The uterus is dissected and the placenta with cotyledons and umbilical cord are separated from the remaining fetal mass and washed in a sterile non-pyrogenic solution containing no antimicrobial agents such as, for example, Lactated Ringers Solution (LRS), as shown in step 26. For the purposes of this application, the terms “placenta-cord fetal component or components” refer to the fetal placenta with cotyledons and umbilical cord which are harvested from the remaining fetal mass for a particular donor mammal while the term “feto-placental unit” includes the dissected placenta-cord fetal component plus the remaining dissected fetal mass. The harvested placenta-cord fetal components are inspected for damage such as, for example, tissue bruising or tearing which may have occurred during harvesting and dissection. Samples of the undamaged harvested placenta-cord fetal components are analyzed for infection using bacterial and/or viral cultures. Undamaged, confirmed non-infected harvested placenta-cord fetal components are weighed and placed in a labeled sterile sample container, as shown in step 28. The sterile sample container holding the placenta-cord fetal components is then transferred into a first clipped, that is, securely closed, sterile container, as shown in step 29 of FIG. 2. Damaged and/or infected dissected placenta-cord components are discarded.

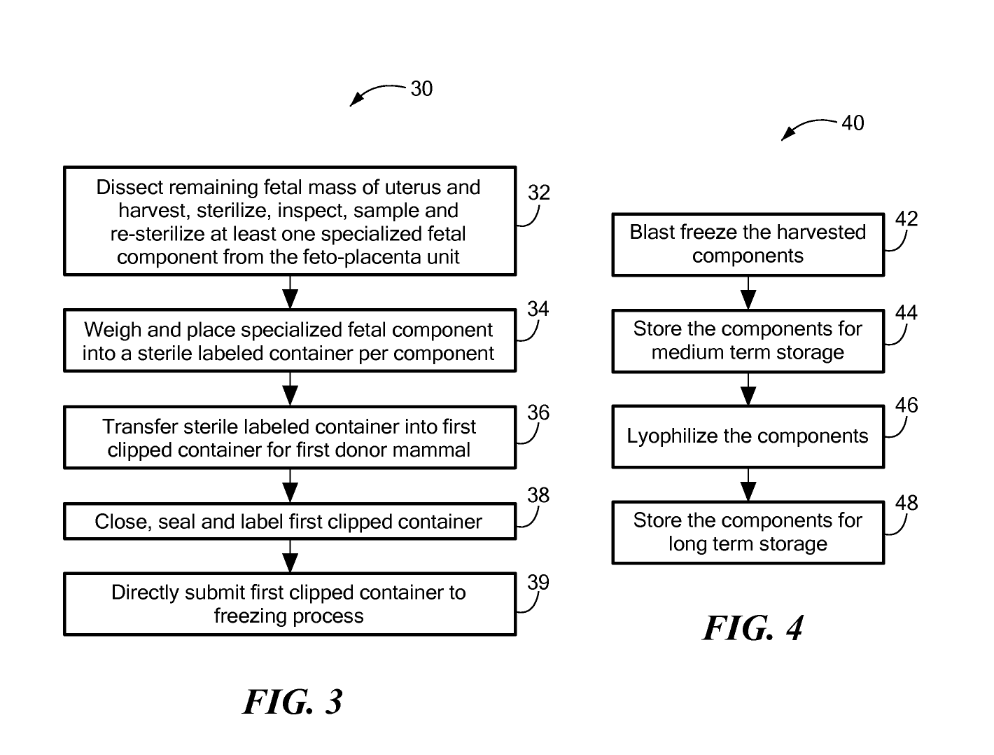

Further embodiments including the steps of method 30 are illustrated in FIG. 3. The remaining fetal mass not including the placenta-cord fetal components is also washed in a sterile non-pyrogenic solution containing no antimicrobial agents such as, for example, LRS, and transferred to a dissection table, as shown in step 32. Under sterile conditions, the sterilized remaining fetal mass is dissected into relevant specialized fetal components such as, for example, a liver component, a spleen component, a whole brain component, an ocular component, a gastro-intestinal component, a female specific component which includes accessory sexual glands, and a male specific component which includes male penile tissue, testicular tissue and accessory sexual glands. The harvested specialized fetal components are inspected for damage during dissection. Samples of undamaged harvested specialized fetal components are analyzed for infection using bacterial and/or viral cultures. Damaged and/or infected dissected specialized fetal components are discarded. Non-damaged, non-infected harvested specialized fetal components are again washed in a similar sterile, non-pyrogenic solution, such as, for example, LRS. Each re-sterilized harvested specialized fetal component is weighed and placed in its own sterile labeled container per component, as shown in step 34. The labeled containers holding the harvested specialized fetal components corresponding to the same first donor mammal are then transferred into the first clipped sterile container along with the placenta-cord fetal components for the same first donor mammal, as shown in step 36.

The first sterile clipped container is closed, sealed and labeled with identification information including weight and any notes regarding the dissection as shown in step 38. The prior multiple inspections of the entire feto-placental unit ensures that normal fetal growth has occurred and that there is no sign of infection and concomitant disease. The first sterile clipped container is directly submitted to a process for freezing the harvested components, as shown in step 39.

The freezing and storage processes 40 of the invention are shown in FIG. 4. The freezing process is accomplished using a fast or rapid freezing otherwise known as a blast freezing, as shown in step 42. The blast freezing lowers the temperature of the components to at least −6° C., and preferably to at least −10° C., and more preferably to at least −20° C., in eight hours, and preferably in six hours, and more preferably in four hours and most preferably in two hours.

After the blast freezing temperature is reached, the components can be stored in a freezer in medium term storage, as shown in step 44, for up to twelve months, and preferably for up to six months, and more preferably for up to three months, and most preferably for up to two months. In medium term storage, the temperature of the components is maintained between −20° C. and −100° C., and preferably between −40° C. and −90° C., and more preferably −60° C. and −80° C., and most preferably between −65° C. and −75° C.

Immediately following blast freezing, or following blast freezing and medium term storage as shown in step 44 of FIG. 4, the components can be further lyophilized or freeze dried to remove water from the components, as shown in step 46, using methods known to those of ordinary skill in the art. A freeze dried form of the component is thereby formed. Lyophilization is conducted on a batch basis by weight and according to type of component. Thus, the placenta-cord fetal component and the specialized liver, spleen, brain, ocular, gastro-intestinal, and female and male specific fetal components are lyophilized by weight according to component type to satisfy minimum weight requirements. Components are lyophilized to weigh a minimum of 10 grams of specific tissue, and preferably a minimum of 8 grams of specific tissue, and more preferably a minimum of 6 grams of specific tissue, and most preferably a minimum of 4 grams of specific tissue.

The components can then be stored for long term storage in their lyophilized, that is, freeze-dried form, as shown in step 48. The temperature of long term storage is maintained between 5° C. and 30° C., and preferably between 10° C. and 25° C., and more preferably between 15° C. and 20° C., and most preferably at 20° C. The duration of long term storage can equal as much as 100 years, and preferably a maximum of 50 years, and more preferably a maximum of 10 years, and even more preferably a maximum of 2 years, and most preferably a maximum of 1 year.

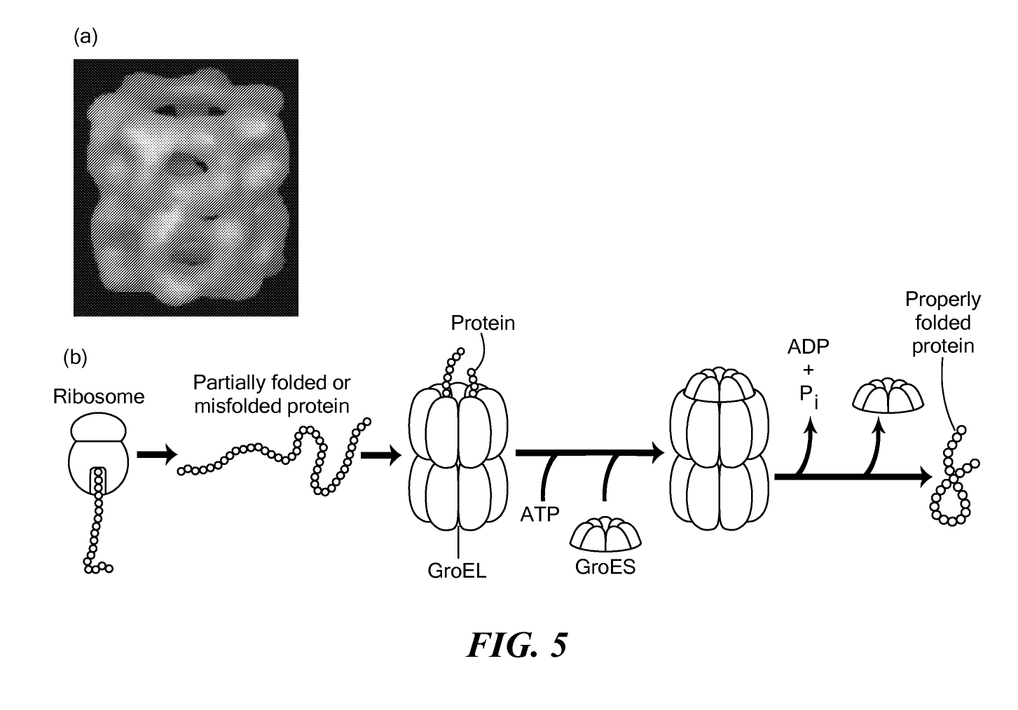

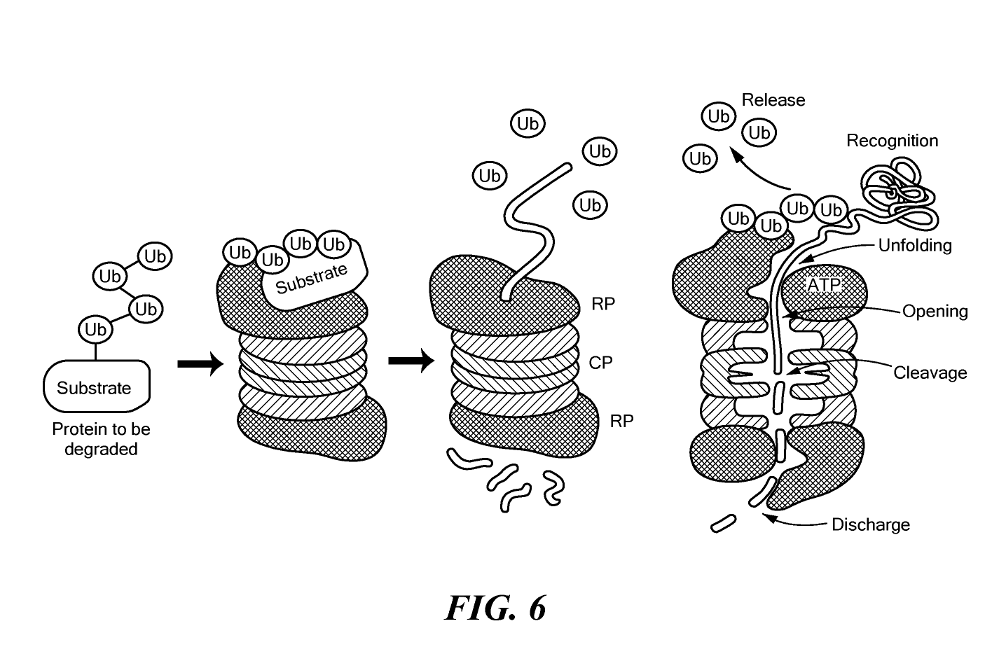

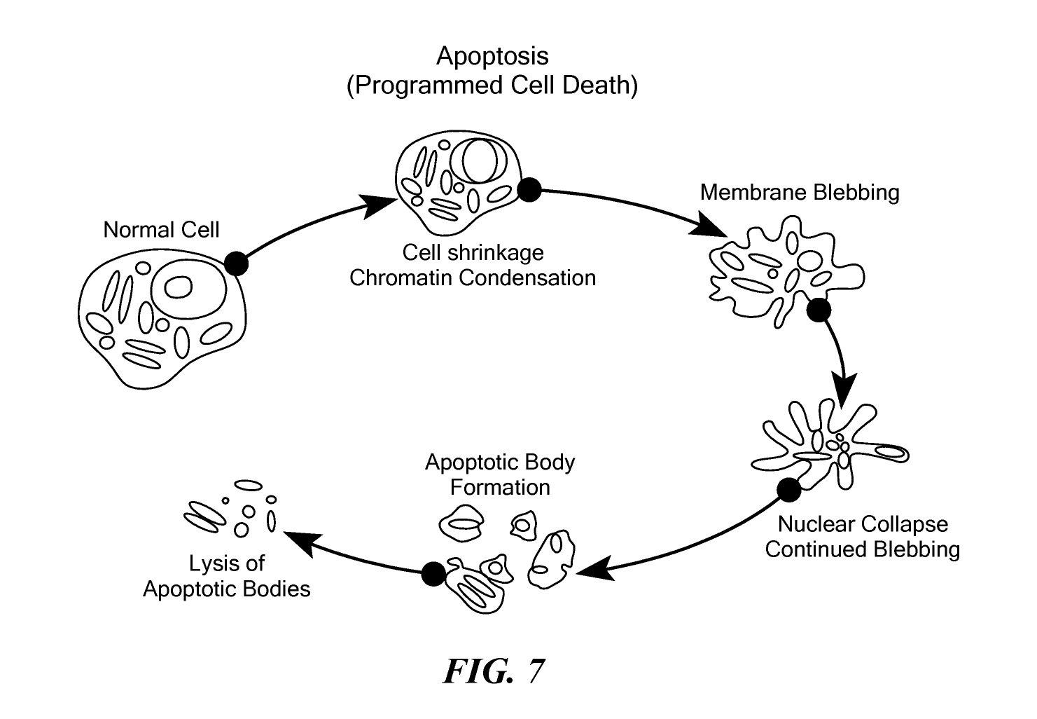

The harvested and processed mammalian fetal unit components include a plurality of proteins or essential fragments thereof. In one embodiment, the mammalian feto-placental unit protein or essential fragments include chaperone proteins. The chaperone proteins can have the ability to re-fold wrongly folded proteins as shown in FIG. 5. In addition or alternatively, the chaperone proteins can degrade otherwise not fully metabolized proteins. FIG. 6 demonstrates this mechanism, where UB identifies ubiquitors, CP identifies chaperone proteins, and RP identifies residual proteins. Furthermore, in addition or alternatively, the chaperone proteins can reverse cellular apoptosis, as shown in FIG. 7. As a cell descends into the spiral of cell death, the cell most commonly goes through a biologic sequence of cellular shut down mechanisms. The shutdown mechanisms take various forms but in essence lead to intracellular protein accumulation mainly through a lack of protein transport out of the cell. The protein accumulation in turn leads to osmotic imbalance, cell membrane disruption and cell membrane disintegration. Chaperone proteins can work intracellularly to fold, refold, transport or degrade accumulated proteins. The reverse of toxic protein accumulation ameliorates osmotic imbalance thereby preventing cellular membrane disruption. As a result, the cellular death spiral is reversed. Further, in addition or alternatively, the chaperone proteins can affect intracellular signaling by controlling conformational changes required for activation or deactivation of signaling proteins, and their assembly in specific signalosome complexes. See Tarone at abstract. Accordingly, in embodiments of the method of the invention, the proteins of the components of the mammalian feto-placental unit can be used for medicinal purposes in the treatment of disease and/or aging in mammalian subjects including, for example, humans. Most disease and aging is characterized by some form of cellular dysfunction including the slowing of cellular mechanisms and the buildup of dysfunctional proteins within the cell. This cellular dysfunction leads to cellular under performance and clinical manifestation of disease and/or aging. The proteins of the components of the mammalian placental unit including essential fragments and chaperone proteins can be administered to promote folding, refolding, transport or degradation of accumulated proteins within cells.

An example of a disease mechanism which can be targeted by the method of the invention is the manifestation of type 2 diabetes. Although this disease can have multi-centric etiologies and manifestations, the cellular basis of the disease relates to the compromised ability of the cell to make insulin receptors and/or to respond to insulin/insulin receptor interaction because of the accumulation of proteins. The compromised cellular abilities lead to a lack of systemic glucose regulation which in turn leads to a cascade of multi-compartmental manifestations of clinical symptoms, but most specifically to an interference with arteriole circulation. The arteriole circulation interference leads to a decrease in blood circulation, vascular constriction, rise in blood pressure, and a decrease in vascular support for organs and cells. Ischemia, cellular apoptosis and eventually cell death result.

The method of the present invention including the administration of mammalian feto-placental unit proteins to a mammalian subject such as a human can counteract and at least partially reverse this cascade of events. In this example, the chaperone proteins administered to the human subject are from an exogenous biologic source of mammalian feto-placental tissue. After administration to the subject, the mammalian feto-placental unit proteins including chaperone proteins can work intracellularly to fold, refold, transport, and/or degrade accumulated proteins. These mechanisms at least partially reverse the toxic accumulation proteins. As a result, the cellular death spiral is reversed at least partially leading to the return of cellular function related to the response of the insulin/insulin receptor complex, the relaxation of arterioles, the decrease in blood pressure, the re oxygenation of tissue, the reverse of ischemia and the return to normal cell function and organ function.

At least portions of the tissues including the proteins can be extracted from the blast frozen components of the mammalian feto-placental unit, thawed and directly administered to the mammalian subject using different procedures employing, for example, a spatula, a sponge, a gel, and encapsulation. Non-limiting procedures for protein administration to a mammalian subject include a sublingual procedure, an intra-ocular procedure, an intra-rectal procedure and an intra-gastro-intestinal procedure.

Alternatively, at least portions of tissues including the proteins can be extracted from the lyophilized components of the mammalian feto-placental unit and reconstituted with a fluid, such as, for example, sterilized water or saline. The reconstituted proteins can be administered to the mammalian subject using various procedures such as, for non-limiting examples, an oral administration, a rectal administration, a cutaneous administration, a subcutaneous administration, an intravenous injection, and an intramuscular injection.

The proteins including the essential fragments thereof harvested from the mammalian feto-placental unit, processed and stored according to a method of the invention are included in the list provided in Table 1. All versions of the database: the uniprot-_{—}20130128_{—}5wcYYr database are included for the purposes of the specification.

Examples of various diseases and/or symptoms characteristic of aging which can be treated by the proteins including the essential fragments thereof harvested from the mammalian feto-placental unit, and processed according to a method of the invention include, for example, type 2 diabetes, type 1 diabetes, cardiomyopathy, prostate cancer, renal dysfunction, and intra- and extra-cellular storage disease, Alzeimer disease, Parkinson disease, sexual dysfunction including female dysfunction (e.g., fluid reduction) and male dysfunction (e.g., erectile dysfunction), hormonal imbalance including hormonal imbalance around the cessation of menus or menopause, musculoskeletal disorder, and neoplasia. For the purposes of this application, “intra- and extra-cellular storage disease” refers to conditions where the body makes proteins but does not have the mechanism such as, for example, the enzymes, to break down the proteins, and the accumulation of such proteins becomes harmful for the subject. Selected proteins are known to those of ordinary skill in the art to ammeliorate selected specific diseases as discussed in, for example, Growth charts for patients with Hunter syndrome, Patel P, Suzuki Y, Maeda M, Yasuda E, Shimada T, Orii KE, Orii T, Tomatsu S., Mol Genet Metab Rep. 2014;1:5-18, PMID: 24955330, [PubMed], and other articles identified in http://www.ncbi.nlm.nih.gov/pubmed/?term=mucopolysaccharide+storage+disease. Accordingly, with respect to the treatment of extra-cellular storage diseases, in another embodiment of the method of the invention, mammalian feto-placental unit proteins are administered to a mammalian subject for the treatment of extracellular storage diseases involving the toxic accumulation of extra-cellular proteins. The mammalian feto-placental proteins act to reduce the toxic accumulation of extra-cellular proteins by degrading, folding, refolding and/or transferring the toxic proteins across cellular membranes. In still other embodiments of the method of the invention, mammalian feto-placental unit proteins are administered to a mammalian subject for the treatment of intra- and extra-cellular storage diseases involving the toxic accumulation of mucopolysaccharides. Mucopolysaccharides, also known as glycosaminoglycans, consist of long chains of sugar molecules. In a normally functioning individual, enzymes are produced to break down the mucopolysaccharides into simpler molecules which the body can then utilize. In individuals suffering from mucopolysaccharide storage diseases, either the individuals do not produce sufficient quantities of the necessary enzymes to break down the mucopolysaccharides into smaller molecules for use by the body, or the individual produces enzymes which are defective and unable to break down the mucopolysaccharides into the necessary smaller molecules. Thus, mucopolysaccharides can accumulate in cells, connective tissue and/or the blood stream and lead to severe disease and death. Such individuals are traditionally treated with exogenous enzymatic therapies for breaking down the mucopolysaccharides. In the methods of the present invention, mammalian feto-placental protecins can be administered to the mammalian subject, and the mammalian feto-placental proteins can break down the mucopolysaccharides into smaller molecules thereby reducing the toxic accumulation of such long chain sugars.

In another aspect, the invention provides a composition including proteins originating from at least one blast frozen, lyophilized component of a harvested mammalian feto-placental unit. In one embodiment, the proteins include at least one essential fragment of at least one of the proteins. In another embodiment, the proteins include chaperone proteins. In yet other embodiments, the component is selected from the group consisting of at least one of a placenta-cord fetal component, a liver component, a spleen component, a whole brain component, an ocular component, a gastro-intestinal component, a female specific component, and a male specific component.

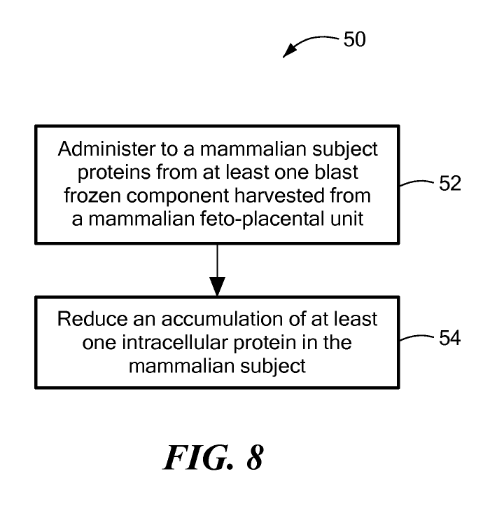

An additional aspect of the invention is shown by method 50 in FIG. 8 which provides for the treatment of a disease or aging in a mammalian subject. The method includes administering to the mammalian subject a plurality of proteins from at least one blast frozen component harvested from a mammalian feto-placental unit, as shown in step 52; and reducing an accumulation of at least one intracellular protein in the mammalian subject, as shown in step 54. In one embodiment, the step of reducing the accumulation of the intracellular protein includes at least one of folding, refolding, transfer, and degradation of the intracellular protein.

### EXAMPLE

A donor sheep certified according to New Zealand Protocol was selected. Placenta-cord, liver, gastro-intestinal and specific male fetal components were dissected, harvested, and processed including inspection, sterilization, blast freezing followed by lyophilization. The lyophilized components were stored for at least of two years. Tissue samples were then extracted from the lyophilized components periodically according to the patient treatment protocol and reconstituted with sterilized saline to form 6 ml injection samples including 1 gram of reconstituted lyophilized component per each sample.

At the onset of treatment, Patient X was in his early 90's and had concomitant prostatic cancer which exhibited as an enlargement of the prostate gland coupled with a prostate specific antigen (PSA) score of over 20. The patient had experienced baldness for approximately 40 years. The patient suffered from clinical stiffness and exhibited stooped posture due to osteoporosis. The patient reported experiencing erectile dysfunction and low libido.

The injection samples including the lyophilized components reconstituted with sterilized saline were injected subcutaneously into Patient X once per month for 6 months and once per 3 months thereafter for 5 years.

After two months of treatment, the patient's clinical physician observed a PSA of 15 and a decrease in prostatic gland size to 30% of the pre-treatment size. Subsequently, the patient's PSA dropped to less than 5 and remained less than 5 thereafter. New hair growth was observed on top of the patient's head. The patient no longer suffered from pre-treatment clinical stiffness and maintained an erect posture. The patient reported return of libido and erectile function.

The foregoing examples and detailed description are not to be deemed limiting of the invention which is defined by the following claims. The invention is understood to encompass such obvious modifications thereof as would be apparent to those of ordinary skill in the art.

What is claimed is:

1. A method of harvesting, processing and storing a plurality of proteins from a mammalian feto-placental unit comprising:
dissecting a mammalian uterus to harvest at least one component of the mammalian feto-placental unit;
blast freezing the component; and
storing the blast frozen component;
wherein the blast frozen component includes the plurality of mammalian feto-placental unit proteins.

2. The method of claim 1, further comprising:
lyophilizing the blast frozen component to remove at least some water from the blast frozen component thereby creating a freeze-dried form; and
storing the lyophilized component;
wherein the lyophilized component includes the mammalian feto-placental unit proteins.

3. The method of claim 1, wherein the proteins comprise at least one essential fragment of at least one of the mammalian feto-placental unit proteins.

4. The method of claim 1, wherein the mammalian feto-placental unit proteins comprise a plurality of chaperone proteins.

5. The method of claim 1, wherein the mammalian feto-placental unit proteins are selected from the group consisting of Serum albumin, Actin, cytoplasmic 1, I alpha globin, Hemoglobin fetal subunit beta, Vimentin, Beta globin chain, TPM1, Annexin A2, Protein disulfide isomerase family A member 3, Alpha-2-HS-glycoprotein, Fatty acid binding protein 5, Cofilin-1, 78 kDa glucose-regulated protein, Gelsolin isoform b, Beta-A globin chain, Glyceraldehyde-3-phosphate dehydrogenase, Heat shock protein alpha, Heat shock protein 70, Peptidylprolyl isomerase A, 14-3-3 protein zeta/delta, Histone H3, Peroxiredoxin 2, Cathepsin D, Uterine milk protein, Tubulin beta chain, Myosin light chain 6, Endoplasmic reticulum protein 29, Tubulin alpha chain, Solute carrier family 2, facilitated glucose transporter member 1, Alpha-1-antitrypsin transcript variant 1, Heat shock protein 10, Pregnancy-associated glycoprotein 3, Hemoglobin subunit beta, Isocitrate dehydrogenase [NADP] cytoplasmic, Elongation factor-1 alpha, Phosphoglycerate kinase, 14-3-3 protein epsilon, Putative tropomyosin, Tumor protein translationally-controlled 1, Galectin-1, Transaldolase 1, Pregnancy-associated glycoprotein 4, Pregnancy-associated glycoprotein 1, Sodium/potassium-transporting ATPase subunit alpha-1, Lamin B1, pregnancy-associated glycoprotein 6, 14-3-3 protein beta/alpha, metallopeptidase inhibitor 2, fatty acid binding protein 5, Myosin regulatory light chain MRCL3, Transferrin, Enolase 1, Cathelicidin-1, 6-phosphogluconate dehydrogenase decarboxylating, Elongation factor-1 alpha, ATP-citrate synthase, Ribosomal protein S8, Pyruvate kinase, Pre-mRNA splicing factor SRP20-like protein, alpha 2, 5 prime, Malate dehydrogenase, Cystatin-B, Chorionic somatomammotropin hormone, Carbonic anhydrase 2, SLC25A6, Decorin, 60S ribosomal protein L6, Protein disulfide isomerase-associated 4, Pregnancy-associated glycoprotein 11, Prostaglandin F synthase, Integrin beta-1, H+ transporting ATP synthase subunit D, RHOA, Adenylate kinase, Lactate dehydrogenase A, RAB10, Glucose-6-phosphate 1-dehydrogenase, Elongation factor 1-delta, Ribosomal protein S17, Insulin-like growth factor-binding protein-7, Ribosomal protein L19, ATP synthase alpha subunit, RAC1, Calpain II 80 kDa subunit, Secreted phosphoprotein 24, CD9 antigen, Aspartate amino ase, DNA mismatch repair protein MutL, Ribosomal protein S6, 14-3-3 protein gamma LDHA protein, Putative peptidase, Myosin light chain kinase, smooth muscle, cAMP-dependent protein kinase regulatory subunit alpha 1, Elongation factor 1-alpha, Actin, GNAZ, Eukaryotic translation initiation factor 5A, Mitochondrial bifunctional methylenetetrahydrofolate dehydrogenase/cyclohydrolase, Solute carrier family 2, facilitated glucose transporter member 3, Thioredoxin, ADP-ribosylation factor 1, NADH dehydrogenase (Ubiquinone) 1 beta, Proteasome subunit alpha type, 2, Gamma fibrinogen, Putative H-ATPase subunit B, Proteasome subunit alpha type, 60S ribosomal protein L10, 14-3-3 protein sigma, Chaperone protein DnaK, Ribosomal protein s15, Putative uncharacterized protein, Aspartyl-tRNA synthetase, and Proteasome subunit alpha.

6. The method of claim 1, further comprising using at least a portion of the mammalian feto-placental unit proteins for medicinal purposes.

7. The method of claim 6, wherein the using step comprises treating for a disease or aging in a mammalian subject.

8. The method of claim 7, wherein the treating step comprises administering the portion of the mammalian feto-placental unit proteins to the mammalian subject with a sublingual procedure.

9. The method of claim 7, wherein the treating step comprises administering the portion of the mammalian feto-placental unit proteins to the mammalian subject with an intra-ocular procedure

10. The method of claim 7, wherein the treating step comprises administering the portion of the mammalian feto-placental unit proteins to the mammalian subject with an intra-rectal procedure.

11. The method of claim 7, wherein the treating step comprises:
reconstituting the portion of the mammalian feto-placental unit proteins of the lyophilized component with a fluid; and
administering the reconstituted portion of the mammalian feto-placental unit proteins to the mammalian subject.

12. The method of claim 11, wherein the administering step comprises administering the reconstituted portion of the mammalian feto-placental unit proteins to the mammalian subject through a procedure selected from the group consisting of an oral administration, a rectal administration, a cutaneous administration, a subcutaneous administration, an intravenous injection, and an intramuscular injection.

13. The method of claim 1 wherein the component is selected from the group consisting of placenta-cord fetal component, a liver component, a spleen component, a whole brain component, an ocular component, a gastro-intestinal component, a female specific component, and a male specific component.

14. A composition comprising:
a plurality of mammalian feto-placental unit proteins from at least one lyophilized, blast frozen component of a harvested mammalian feto-placental unit reconstituted with a fluid.

15. The composition of claim 14, wherein the mammalian feto-placental unit proteins comprise at least one essential fragment of at least one of the proteins.

16. The composition of claim 14, wherein the mammalian feto-placental unit proteins comprise a plurality of chaperone proteins.

17. The composition of claim 14 wherein the lyophilized, blast frozen component is selected from the group consisting of a placenta-cord fetal component, a liver component, a spleen component, a whole brain component, an ocular component, a gastro-intestinal component, a female specific component, and a male specific component.

18. A method of treatment of a disease or aging in a mammalian subject comprising:
administering to the mammalian subject a plurality of mammalian feto-placental unit proteins from at least one blast frozen component harvested from a mammalian feto-placental unit; and
reducing an accumulation of at least one non-mammalian feto-placental unit protein in the mammalian subject.

19. The method of claim 18, wherein the mammalian feto-placental unit proteins comprise at least one essential fragment of at least one of the mammalian feto-placental unit proteins.

20. The method of claim 18 wherein the mammalian feto-placental unit proteins comprise a plurality of chaperone proteins.

21. The method of claim 18, wherein the step of reducing the accumulation of the intracellular protein comprises at least one of folding of at least a portion of the intracellular protein, refolding of at least a portion of the intracellular protein, degrading at least a portion of the intracellular protein, and transferring at least a portion of the intracellular protein across a cellular membrane.

22. The method of claim 11, wherein the fluid is an oil.

23. The method of claim 12, wherein the procedure is the cutaneous administration procedure.

24. The method of claim 12, wherein the cutaneous administration procedure comprises applying a cutaneous patch including the reconstituted portion of the mammalian feto-placental unit proteins to the mammalian subject.

25. The method of claim 7, wherein the disease is a storage disease involving the toxic accumulation of at least one of an intra-cellular protein and an extra-cellular protein.

26. The method of claim 7, wherein the disease is a storage disease involving the toxic accumulation of at least one of an intra-cellular sugar and an extra-cellular sugar.

27. The method of claim 26, wherein at least one of the intra-cellular sugar and the extra-cellular sugar is a mucopolysaccharide.

28. The composition of claim 14 further comprising a kit,
wherein the kit comprises a patch including at least a portion of the reconstituted mammalian feto-placental unit proteins; and
wherein the patch is adapted for cutaneous administration to a mammalian subject.

29. The method of claim 18, wherein the non-mammalian feto-placental unit protein is at least one of an intra-cellular protein and an extra-cellular protein.

30. A method of treatment of a disease or aging in a mammalian subject comprising:
administering to the mammalian subject a plurality of mammalian feto-placental unit proteins from at least one blast frozen component harvested from a mammalian feto-placental unit; and
reducing an accumulation of a sugar;
wherein the sugar is at least one of an intra-cellular sugar and an extra-cellular sugar.

31. The method of claim 30, wherein the at least one of the intra-cellular sugar and the extra-cellular sugar is a muco-polysaccharide.
